(19) United States
(12) Patent Application Publication
(10) Pub. No.: US 2026/0096808 A1
(43) Pub. Date: Apr. 9, 2026

(54) MODEL-BASED IMAGING IN ULTRASOUND

(71) Applicant: Exo Imaging, Inc., Santa Clara, CA (US)

(72) Inventors: Seshadri SRINIVASAN, Santa Clara, CA (US); Bernal MANZANILLA, Santa Clara, CA (US); Ruiying ZHANG, Santa Clara, CA (US); Biren PARMAR, Santa Clara, CA (US); Sandeep AKKARAJU, Santa Clara, CA (US)

(21) Appl. No.: 19/211,076

(22) Filed: May 16, 2025

Related U.S. Application Data
(60) Provisional application No. 63/649,312, filed on May 17, 2024.

(51) Int. Cl. A61B 8/00

(52) U.S. Cl. CPC A61B 8/5207; A61B 8/5269

(57) ABSTRACT

A final ultrasound image can be formed by receiving an actual ultrasound image from an ultrasound imaging device, the actual ultrasound image being formed using parameters, creating a synthetic ultrasound image using a simulation model based on the parameters; and forming the final ultrasound image based on the synthetic ultrasound image and the actual ultrasound image.

### CROSS-REFERENCE TO RELATED APPLICATIONS

The present application claims priority under 35 U.S.C. 119(e) to, and the benefit of, U.S. Provisional App. No. 63/649,312, filed on May 17, 2024, the entirety of which is incorporated herein by reference.

### TECHNICAL FIELD

The present disclosure relates to systems, devices, and methods for ultrasound imaging, more specifically, to systems and methods for performing model-based imaging in ultrasound imaging.

### BACKGROUND

Ultrasound imaging is an imaging method that uses sound waves to produce images of structures within a patient's body. Because ultrasound images are captured in real-time, they can also show movement of the body's internal organs as well as blood flowing through the blood vessels. The images can provide valuable information for diagnosing and directing treatment for a variety of diseases and conditions.

In the field of medical imaging, ultrasound sound waves have frequencies above those audible to the human ear, that is, greater than approximately 20 MHz. Ultrasound typically used in clinical settings has frequencies between 2 and 12 MHz. Lower frequencies produce less resolution but have greater depth of penetration into the body; higher frequencies produce greater resolution, but depth of penetration is limited. High-frequency ultrasound (HFUS) with its higher resolution is a useful tool in many specialties, which focus on skin evaluations and skin disorders. This trade-off between resolution and penetration is an inherent limit in the quality of ultrasound images, especially images of an anatomy that requires a higher degree of penetration.

### SUMMARY

In accordance with at least some embodiments disclosed herein is the realization that ultrasound systems can use well-established signal and image processing stages on raw channel data for obtaining the ultrasound image. Further, some embodiments disclosed herein also include the realization that these methods do not incorporate comparisons between simulated data of the underlying anatomy and the actual data that is obtained from the anatomy.

Advantageously, some embodiments disclosed herein can therefore provide for an ultrasound system and method that can leverage both high frequency and low frequency ultrasound imaging to form higher quality ultrasound images.

Methods of forming a final ultrasound image are herein disclosed. In accordance with some embodiments, a method comprises, at a computing device of an ultrasound imaging device, receiving an actual ultrasound image from the ultrasound imaging device, the actual ultrasound image being formed using parameters, creating a synthetic ultrasound image using a simulation model based on the parameters; and forming the final ultrasound image based on the synthetic ultrasound image and the actual ultrasound image.

Receiving the actual ultrasound image from the ultrasound imaging device may include receiving the actual ultrasound image from the ultrasound imaging device at a first imaging frequency.

The parameters may include a scatterer intensity and distribution pattern used to form the actual ultrasound image. The scatterer intensity and distribution pattern is a distribution of an amount of ultrasound energy that is scattered or reflected back from an anatomy of interest. The synthesizing may also include training the simulation model by comparing the actual ultrasound image and an initial simulated ultrasound image.

The synthesizing may also include training the simulation model by comparing the scatterer intensity and distribution pattern to expected templates of scatterer intensity and distribution pattern for an anatomy of interest to reduce artifacts, which may include noise.

In some embodiments, the simulation model may comprise a forward propagation model. In some embodiments, the simulation model may comprise an artificial intelligence (AI) model. In some embodiments, the simulation model may comprise a real-time ultrasound model based on a real-time simulation of ultrasound data. In some embodiments, the simulation model may comprise a forward propagation real-time ultrasound model. In some embodiments, the simulation model may comprise a spatial-temporal model. In some embodiments, the spatial-temporal model may comprise used to create lateral flow or anatomic flow instead of axial flow. Lateral flow is a flow that is perpendicular to an ultrasound beam, the anatomic flow is a flow that is neither strictly axial nor strictly lateral to the ultrasound beam, and the axial flow is a flow that is parallel to the ultrasound beam.

In some embodiments, the simulation model may be based on an underlying anatomy of the actual ultrasound image. The simulation model may be specific to an underlying anatomy of the actual ultrasound image.

The parameters may include transducer parameters and ultrasound imaging parameters to form the actual ultrasound image.

In some embodiments, the synthesizing may include adapting the parameters used to form the actual ultrasound image to minimize differences between the actual ultrasound image and an initial simulated ultrasound image. In some embodiments, the synthesizing may include adapting the scatterer intensity and distribution pattern to minimize differences between the actual ultrasound image and the initial simulated ultrasound image. In some embodiments, the synthesizing may include using the adapted scatterer intensity and distribution pattern to form the simulated ultrasound image. In some embodiments, the synthesizing may include adapting the transducer parameters to reduce reverb to minimize differences between the actual ultrasound image and an initial simulated ultrasound image. In some embodiments, the synthesizing may include adapting a lens thickness to reduce reverb to minimize differences between the actual ultrasound image and an initial simulated ultrasound image.

The final ultrasound image may be at a desired resolution different from a resolution of the actual ultrasound image. In some embodiments, the final ultrasound image may be at one or more frequencies different from a frequency of the actual ultrasound image. In some embodiments, the final ultrasound image may be at one or more apertures different from an aperture of the actual ultrasound image. In some embodiments, the final ultrasound image may be at one or more directions different from a direction of the actual ultrasound image. In some embodiments, the synthesizing may include improving penetration of the actual ultrasound image to form the final ultrasound image. In some embodiments, the synthesizing may include improving resolution of the actual ultrasound image to form the final ultrasound image.

The synthesizing may include improving speckle of the actual ultrasound image to form the final ultrasound image. In some embodiments, the synthesizing may include using a higher frame rate than the actual ultrasound image to form the final ultrasound image.

The synthesizing may include displaying the final ultrasound image at a second imaging frequency. In some embodiments, the second imaging frequency may be greater than the first imaging frequency, the actual ultrasound image is at a first spatial resolution, the final ultrasound image is at a second spatial resolution, and the second spatial resolution is higher than the first spatial resolution.

The synthesizing may include reducing or eliminating reverb noise of the final ultrasound image based on the simulation model of an imaging anatomy. In some embodiments, the synthesizing may include removing unwanted aspects from the simulated ultrasound image. In some embodiments, the synthesizing may include removing artifacts that arise from a transducer from the simulated ultrasound image. In some embodiments, the synthesizing may include improving a penetration depth of the simulated ultrasound image at high imaging frequencies. In some embodiments, the synthesizing may include training the simulation model with time to create a spatial-temporal model. In some embodiments, the synthesizing may include improving a doppler image quality of the final ultrasound image.

The synthetic ultrasound may be a scatterer model.

The synthesizing may include improving a needle detection of the final ultrasound image. In some embodiments, the synthesizing may include rendering the final ultrasound image using motion mode. In some embodiments, the synthesizing may include training the simulation model by automatically identifying and adapting imaging presets by comparing one or more of the simulation models with one or more templates of stored models.

A method of processing an ultrasound image is herein disclosed. In some embodiments, the method may include receiving the ultrasound image from an anatomy of interest, and thereafter training a model based on the anatomy of interest by minimizing a difference between sample ultrasound images and expected ultrasound images. An enhanced ultrasound image can then be generated via the trained model from the ultrasound image.

An ultrasound imaging device is herein disclosed. The ultrasound imaging device may be configured to receive an actual ultrasound image from the ultrasound imaging device. In some embodiments, the actual ultrasound image can be formed using one or more parameters. Further, a synthetic ultrasound image can be created using a simulation model based on the parameters. Thereafter, the final ultrasound image can be based on the synthetic ultrasound image and the actual ultrasound image.

For example, the ultrasound imaging device may include a computing circuit, one or more processors, memory, and one or more programs stored in the memory and configured for execution by the one or more processors, the one or more programs comprising instructions for generating the final ultrasound image.

A non-transitory computer readable storage medium is herein disclosed. The medium can store one or more programs that, when executed by a computing device having one or more processors and memory, cause the computing device to perform operations that may include: receiving an actual ultrasound image from an ultrasound imaging device, the actual ultrasound image being formed using parameters; and creating a synthetic ultrasound image using a simulation model based on the parameters; and/or forming the final ultrasound image based on the synthetic ultrasound image and the actual ultrasound image.

A non-transitory computer readable storage medium is herein disclosed, which may store one or more programs that, when executed by a computing device, can cause the computing device to perform operations including any of the steps disclosed in any of the preceding paragraphs.

A method is herein disclosed, which may include any of the steps disclosed in any of the preceding paragraphs.

A system is herein disclosed, which may include one or more devices configured to perform any of the methods disclosed in any of the preceding paragraphs.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

The systems, methods and devices of this disclosure each have several innovative aspects, no single one of which is solely responsible for the desirable attributes disclosed herein.

It is to be understood that both the foregoing general description and the following detailed description are exemplary and explanatory and are intended to provide further explanation of the subject technology.

Note that the various embodiments described above can be combined with any other embodiments described herein. The features and advantages described in the specification are not all inclusive and, in particular, many additional features and advantages will be apparent to one of ordinary skill in the art in view of the drawings, specification, and claims. Moreover, it should be noted that the language used in the specification has been principally selected for readability and instructional purposes and may not have been selected to delineate or circumscribe the inventive subject matter.

### BRIEF DESCRIPTION OF THE DRAWINGS

For a better understanding of the various described implementations, reference should be made to the Description of Implementations below, in conjunction with the following drawings in which like reference numerals refer to corresponding parts throughout the figures. The illustrated embodiments are intended to illustrate, but not to limit, the inventions.

FIG. 1 illustrates an exemplary workflow for model-based imaging in ultrasound, in accordance with some embodiments.

FIG. 2 illustrates an ultrasound system for imaging a patient, in accordance with some embodiments.

FIG. 3 illustrates a block diagram of an exemplary ultrasound device, in accordance with some embodiments.

FIG. 4 illustrates a block diagram of a computing device, in accordance with some embodiments.

FIGS. 5A, 5B, and 5C are examples of an actual ultrasound image, a simulated ultrasound image, and a synthesized ultrasound image, in accordance with some embodiments.

Reference will now be made to implementations, examples of which are illustrated in the accompanying drawings. In the following description, numerous specific details are set forth in order to provide a thorough understanding of the present invention. However, it will be apparent to one of ordinary skills in the art that the present invention may be practiced without requiring some of these specific details.

### DETAILED DESCRIPTION

Reference will now be made in detail to implementations, examples of which are illustrated in the accompanying drawings. In the following detailed description, numerous specific details are set forth in order to provide a thorough understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

A model-based ultrasound imaging systema and method are herein disclosed. Discussion herein relating to “an anatomy” or “the anatomy” should be understood to refer to any portion of the anatomy, e.g., human anatomy, upon which an ultrasound system may be focused or directed towards at any given time.

In an embodiment, a model of the anatomy may be created. In some embodiments, the model of the anatomy (such as the scatterer distribution and the scatterer intensity), use a real-time simulation of the ultrasound data, adapt/evolve the model by comparing the expected and actual data, and subsequently representing a “new” or “different” ultrasound image based on the model and the ultrasound simulation.

Using a model of the anatomy and a real-time simulation of the ultrasound data, can have may applications. A non-exhaustive list of applications for model-based imaging is included here. The use of a model-based method of ultrasound imaging may also have other applications not listed herein.

In some embodiments, a model-based method of ultrasound imaging, as disclosed herein, may be used to display a high-resolution image, e.g., a high spatial resolution image, despite a low frequency imaging condition being necessary to achieve penetration. In some embodiments, a model-based method of imaging may also be used to reduce or eliminate noise, such as reverb noise, from the model. In some embodiments, a model-based method of imaging may also be used to removed unwanted aspects of an image, such as artifacts, including artifacts or other unwanted aspects of an image that may arise from a transducer defect. In some embodiments, a model-based method of imaging may also be used to improve penetration at high imaging frequencies. A model-based method of imaging may also be used to evolve the model over time, to improve the model, e.g., by creating a spatio-temporal model. In some embodiments, a model-based method of imaging may also be used to improve doppler image quality. In some embodiments, a model-based method of imaging may also be used to identify and adapt, e.g., automatically, imaging presets. This may be accomplished by comparing one or more models with templates of stored models.

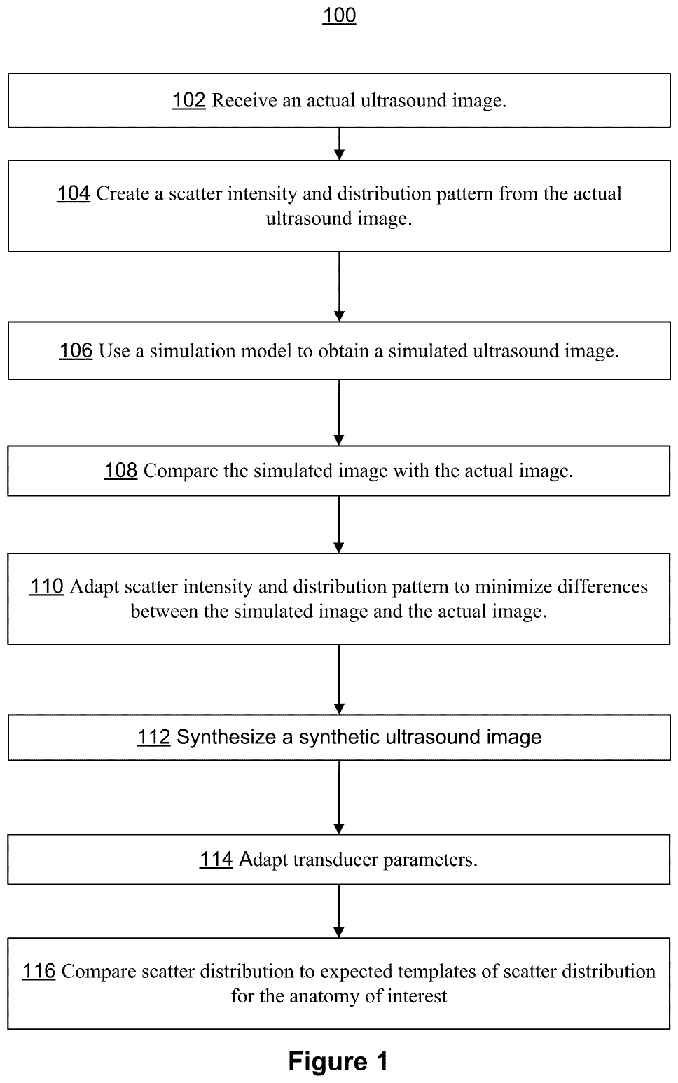

FIG. 1 illustrates an exemplary workflow 100 for improving image quality of an ultrasound imaging device based on a model using a simulated image, in accordance with some embodiments.

In step 102, an actual ultrasound image can be obtained. The actual ultrasound image may be obtained at some desired imaging frequency, which may in some cases be related to the anatomy to be imaged, e.g., how much penetration is needed.

In step 104, a scatterer intensity and/or distribution pattern can be created from the actual ultrasound image. In ultrasound imaging, “scattering” occurs when a sound wave strikes a structure with a different acoustic impedance to the surrounding tissue, and which is smaller than the wavelength of the incident sound wave. Such structures are known as “diffuse reflectors,” in contrast with “specular reflectors” which have smooth interfaces. Diffuse reflectors may cause ultrasound waves to scatter in all directions, resulting in multiple echoes propagating from small structures in the body.

Scattering may result in echoes with smaller amplitudes, and the echoes may also interact with each other. Interaction of echoes with each other may cause interference in the waves, which may be constructive or destructive. Variations in scatterer intensity may result in speckled images, which may be seen as irregularities when viewing a resulting image, such as grainy appearance. Scatterer may be more or less intense, depending on how specular or diffuse the anatomy being scanned is. Scatterer distribution may also vary between different tissue types, which may in some use cases be found proximate to each other during a scan. A scatterer intensity and distribution pattern is a distribution of an amount of ultrasound energy that is scattered or reflected back from an anatomy of interest.

In step 106, a simulation model may be used to obtain a simulated ultrasound image. In some embodiments, the simulation model may be an artificial intelligence model. In some embodiments, the simulation model may use forward propagation, which is the feeding of input data in the forward direction, through the network, to generate an output.

In some embodiments, the simulation model may be a real-time ultrasound model based on a real-time simulation of ultrasound data. In some embodiments, the simulation model may be a forward propagation real-time ultrasound model. In some embodiments, the simulation model may be implemented by or as a neural network and may use artificial intelligence and/or machine learning.

In some embodiments, the simulation model may be a spatial-temporal model. In some embodiments, the simulation model may become a spatial-temporal model after it is trained over time. In some embodiments, the spatial-temporal model may be used to create lateral flow, which is a flow that is perpendicular to an ultrasound beam. In some embodiments, the spatial-temporal model may be used to crate anatomic flow, which is flow that is neither strictly axial nor strictly lateral to the ultrasound beam. In some embodiments, lateral flow or anatomic flow may be used instead of axial flow, which is flow that is parallel to the ultrasound beam.

In accordance with one embodiment, the simulation model may create the simulated ultrasound image by feeding, into the model, parameters, which may include transducer parameters and/or ultrasound imaging parameters, that are the same as or similar to those used to obtain the actual ultrasound image obtained in step 102. In some embodiments, transducer parameters may be adapted, e.g., to reduce reverb to minimize differences between the actual ultrasound image and the initial simulated ultrasound image.

In some embodiments, the simulation model may be based on an underlying anatomy of the actual ultrasound image. In some embodiments, the simulation model may be specific to an underlying anatomy of the actual ultrasound image.

In step 108, the simulated image, as generated in step 106 by the simulation model, and the actual image, as generated conventionally in step 102, are compared. Comparison may be accomplished by methods such as using a mean squared error or L1 norm on selected regions of the actual images or filtered versions of the images. Other comparison methods may also be used.

In step 110, the scatterer intensity and distribution pattern, as created in step 104, may be adapted, to minimize the differences between the simulated and actual image. In some embodiments, adaptation may include variation of strength of the scatterer and/or density of the scatterer distribution. For example, for specular reflectors, the strength of the distribution may be changed and for speckle targets, the number of scatterers and the distribution may be changed. In some embodiments, the scatterer intensity and distribution pattern may be compared to expected templates of scatterer intensity and distribution pattern for the anatomy of interest, e.g., the anatomy being scanned. In some embodiments, this may reduce artifacts, such as noise or reverb.

In step 112, the computed scatterer intensity and distribution may be used to synthesize one or more “synthetic” ultrasound images at any desired resolution. The computed scatterer intensity and distribution may in some embodiments be a result or product of the adaption of the scatterer intensity and distribution pattern, discussed above, which minimized differences. The “synthetic” ultrasound image may also be referred to herein as a “final” ultrasound image, which may take into account the actual ultrasound image and the simulated ultrasound image. In some embodiments, the synthetic ultrasound image may have improved penetration compared to the actual ultrasound image. In some embodiments, penetration depth may be improved at high frequencies. In some embodiments, the synthetic image may have improved resolution compared to the actual ultrasound image. In some embodiments, the synthetic image may have improved speckle compared to the actual ultrasound image. In some embodiments, the synthetic image may have a different, e.g., higher, frame rate, compared to the actual ultrasound image. In some embodiments, the synthetic image may have improved doppler image quality compared to the actual ultrasound image. In some embodiments, the synthetic image may have improved needle detection quality. In some embodiments, the synthetic image may be rendered using motion mode.

In some embodiments, the one or more synthetic ultrasound images can incorporate multiple frequencies, multiple apertures, multiple directions, etc., to create better penetration or better resolution or reduced speckle or improved penetration, etc.

In some embodiments, the final ultrasound image is at a resolution different from the resolution of the actual ultrasound image. In some embodiments, the final ultrasound image is at a frequency, or more than one frequency, different from the frequency of the actual ultrasound image. In some embodiments, the final image may be displayed at a second frequency. In some embodiments, the second frequency may be higher, and the resolution at which the final image is displayed. In some embodiments, the final ultrasound image is at an aperture, or more than one aperture, different from the aperture of the actual ultrasound image. In some embodiments, the final ultrasound image is at a direction, or more than one direction, different from a direction of the actual ultrasound image. In some embodiments, the synthesized image will have fewer artifacts, or other unwanted items, compared to the actual ultrasound image.

In step 114, transducer parameters may be adapted, such as adapting a lens thickness to reduce reverb. Adapting the parameters used to form the actual ultrasound image may be done in order to minimize differences between the actual ultrasound image and an initial simulated ultrasound image. The model can take into account an ideal transducer that does not have artefacts such as reverb. Accordingly, step 114 may be seen as design aspects of the model. Optionally, step 114 may take place after the synthetic ultrasound image is synthesized in step 112, as discussed above.

In step 116, scatterer distribution may be compared to expected templates of scatterer distribution for the anatomy of interest. This may be done after a synthetic image in synthesized in step 112. Comparing the scatterer distribution to expected scatterer distribution templates may be helpful in reducing artifacts, such as noise. For example, ultrasound data from imaging of bones or air or other gasses in the body is largely noise because bones and gasses do not conduct sound well. In accordance with some embodiments disclosed herein is the realization that it would be helpful to remove this noise using a comparison between scatterer distribution and expected scatterer distribution, e.g., for bones or gasses.

In some examples, the ultrasound imaging device includes the transducers that may be piezoelectric micromachined ultrasonic transducers (PMUT). In some examples, the transducers may be ultrasound transducers that may handle both transmission and reception. In some examples, the transducers may be a matrix of transducer array that may be driven by amplifiers used to drive columns of imaging pixels or columns of the transducers independently.

The amplifiers and transducers may be integrated on an application specific integrated circuit (ASIC). The amplifiers and transducers may be integrated on field programmable gate arrays (FPGAs). In some examples, beamforming may be done on integrated circuits such as ASIC and FPGA coupled to or integrated with the transducers. In some examples, a computing device coupled to the integrated circuit of the transducers may be utilized to further process the ultrasound image. The computing device may be a mobile phone, a personal computer, a server, or a cloud computing network, etc.

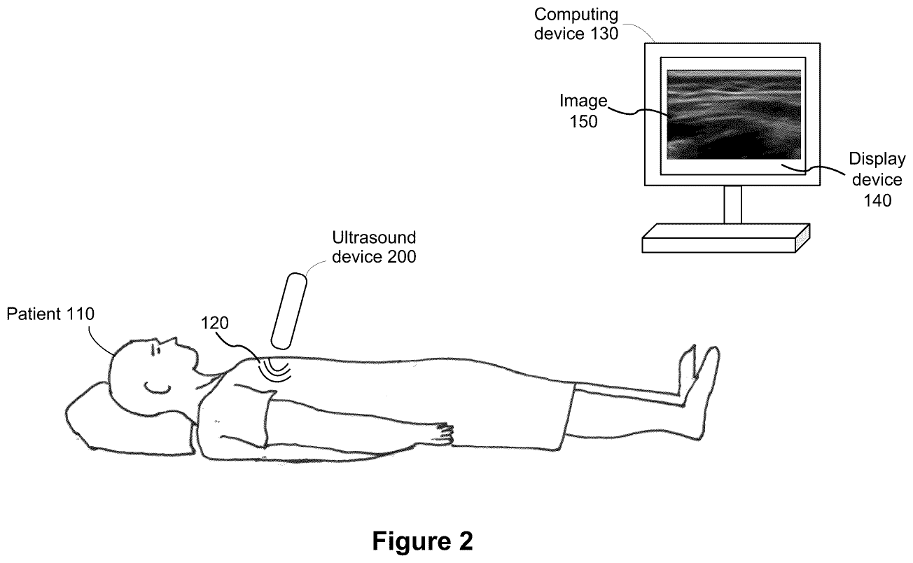

FIG. 2 illustrates an ultrasound system for imaging a patient, in accordance with some embodiments.

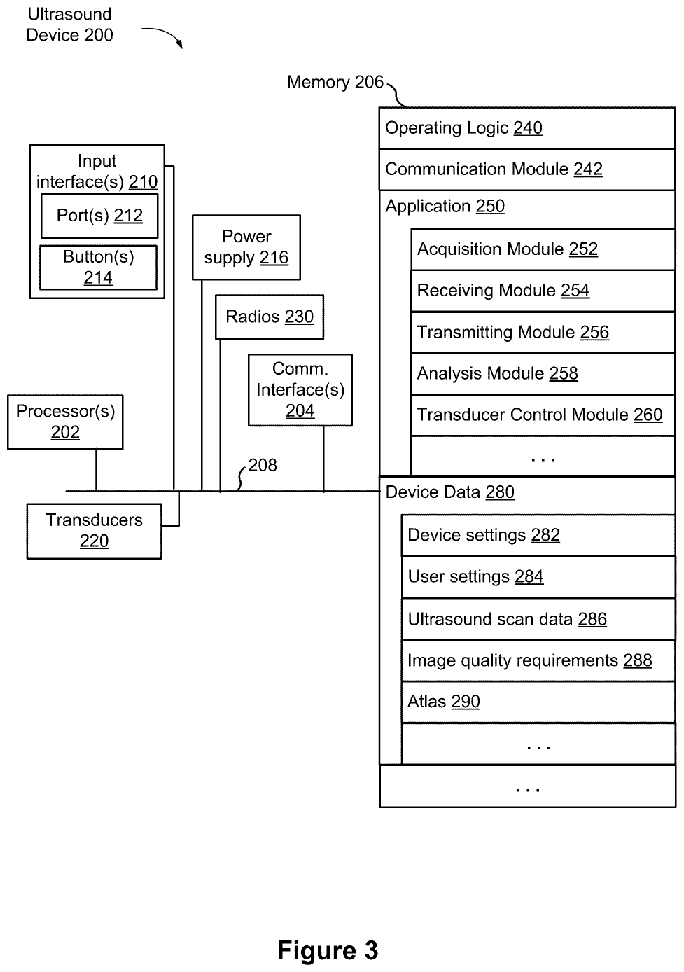

In some embodiments, an ultrasound device 200 is a portable, handheld device. The ultrasound device 200 can include a probe portion that includes transducers (e.g., transducers 220, FIG. 3). The transducers can be arranged in an array.

In some embodiments, the ultrasound device 200 includes an integrated control unit and user interface. The ultrasound device 200 can include a probe that communicates with a control unit and user interface that is external to the housing of the probe itself.

During operation, the ultrasound device 200 (e.g., via the transducers) can produce sound waves 120 that are transmitted toward an organ, such as a heart or a lung, of a patient 110. The internal organ, or other object(s) to be imaged, may reflect a portion of the sound waves toward the probe portion of the ultrasound device 200, which are received by the transducers 220. In some embodiments, the ultrasound device 200 transmits the received signals to a computing device 130, which uses the received signals to create an image 150 that is also known as a sonogram. The computing device 130 can include a display device 140 for displaying ultrasound images, and other input and output devices (e.g., keyboard, touch screen, joystick, touchpad, and/or speakers).

FIG. 3 illustrates a block diagram of an exemplary ultrasound device 200, in accordance with some embodiments.

In some embodiments, the ultrasound device 200 includes one or more processors 202, one or more communication interfaces 204 (e.g., network interface(s)), memory 206, and/or one or more communication buses 208 for interconnecting such components (which may be referred to as a “chipset”).

In some embodiments, the ultrasound device 200 can include one or more input interfaces 210 that facilitate user input. For example, in some embodiments, the input interfaces 210 include port(s) 212 and button(s) 214. In some embodiments, the port(s) can be used for receiving a cable for powering or charging the ultrasound device 200, or for facilitating communication between the ultrasound device and other devices (e.g., computing device 130, computing device 300, display device 140, printing device, and/or other input output devices and accessories).

In some embodiments, the ultrasound device 200 includes a power supply 216. For example, the ultrasound device 200 can be battery-powered. In some embodiments, the ultrasound device can be powered by a continuous AC power supply.

In some embodiments, the ultrasound device 200 includes a probe portion that includes transducers 220, which may also be referred to as transceivers or imagers. Examples of transducers 220 include, without limitation, piezoelectric micromachined ultrasonic transducers (PMUT) and capacitive micromachined ultrasonic transducers (CMUT).

In some embodiments, the transducers 220 can be based on photo-acoustic or ultrasonic effects. For ultrasound imaging, the transducers 220 transmit ultrasonic waves towards a target (e.g., a target organ, blood vessels, etc.) to be imaged. The transducers 220 receive reflected sound waves (e.g., echoes) that bounce off body tissues. The reflected waves are then converted to electrical signals and/or ultrasound images.

In some embodiments, the probe portion of the ultrasound device 200 can be housed separately from the computing and control portion of the ultrasound device. For example, the probe portion of the ultrasound device 200 can be integrated in the same housing as the computing and control portion of the ultrasound device 200.

Further, in some embodiments, part of the computing and control portion of the ultrasound device can be integrated in the same housing as the probe portion, and part of the computing and control portion of the ultrasound device is implemented in a separate housing that is coupled communicatively with the part integrated with the probe portion of the ultrasound device. In some embodiments, the probe portion of the ultrasound device can have a respective transducer array that is tailored to a respective scanner type (e.g., linear, convex, endocavitary, phased array, transesophageal, 3D, and/or 4D). In the present disclosure, “ultrasound probe” may refer to the probe portion of an ultrasound device, or an ultrasound device that includes a probe portion.

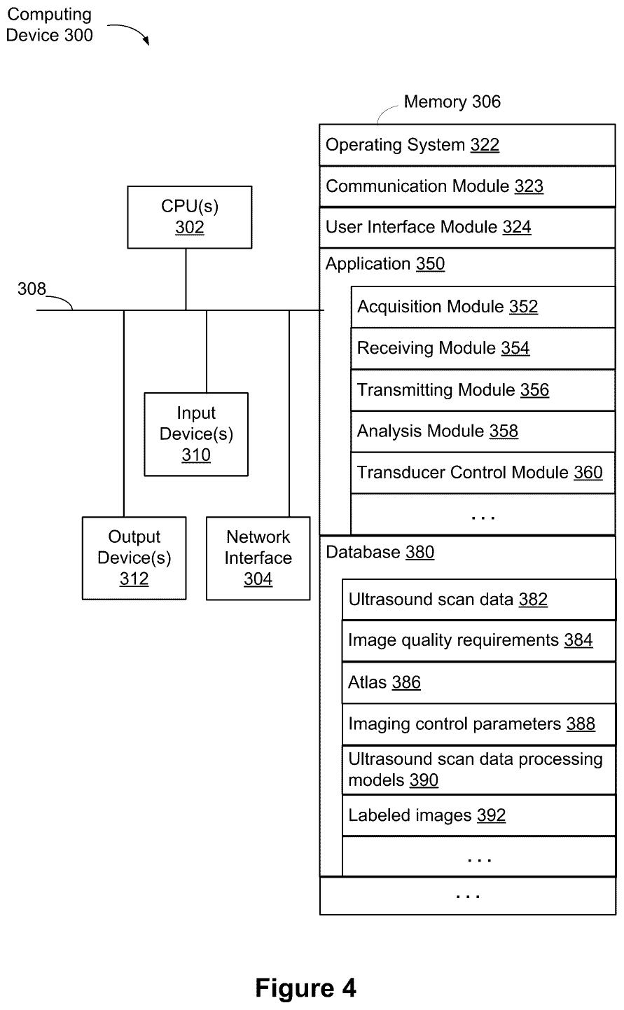

In some embodiments, the ultrasound device 200 can include radios 230. The radios 230 enable one or more communication networks and/or allow the ultrasound device 200 to communicate with other devices, such as the computing device 130 in FIG. 2, the display device 140 in FIG. 2, and/or the computing device 300 in FIG. 4. In some implementations, the radios 230 are capable of data communications using any of a variety of custom or standard wireless protocols (e.g., IEEE 802.15.4, Wi-Fi, ZigBee, 6LoWPAN, Thread, Z-Wave, Bluetooth Smart, ISA100.5A, WirelessHART, MiWi, Ultrawide Band (UWB), software defined radio (SDR), etc.) custom or standard wired protocols (e.g., Ethernet, HomePlug, etc.), and/or any other suitable communication protocol, including communication protocols not yet developed as of the filing date of this document.

The memory 206 can include high-speed random-access memory, such as DRAM, SRAM, DDR RAM, or other random-access solid-state memory devices. Optionally, the memory 206 can include non-volatile memory, such as one or more magnetic disk storage devices, one or more optical disk storage devices, one or more flash memory devices, or one or more other non-volatile solid state storage devices. The memory 206, optionally, includes one or more storage devices remotely located from one or more processor(s) 202. The memory 206, or alternatively the non-volatile memory within the memory 206, includes a non-transitory computer-readable storage medium.

In some implementations, the memory 206 or the non-transitory computer-readable storage medium of the memory 206 (either or both of which may be referred to herein as “memory”) can store various programs, modules, and data structures.

For example, the memory can store programs, modules, and data structures, or a subset or superset thereof, such as operating logic 240, a communication module 242, an application 250, and/or a data device 280.

The operating logic 240 can include procedures for handling various basic system services and for performing hardware dependent tasks.

The communication module 242 (e.g., a radio communication module) can be configured to connect to and communicate with other network devices (e.g., a local network, such as a router that provides Internet connectivity, networked storage devices, network routing devices, server systems, computer device 130, computer device 300, and/or other connected devices, etc.) coupled to one or more communication networks via the communication interface(s) 204 (e.g., wired or wireless).

The application 250 can be configured to acquire ultrasound data (e.g., imaging data) of a patient, and/or to control one or more components of the ultrasound device 200 and/or other connected devices (e.g., in accordance with a determination that the ultrasound data meets, or does not meet, certain conditions).

In some embodiments, the application 250 can be configured to include an acquisition module 252, a receiving module 254, a transmitting module 256, an analysis module 258, and/or a transducer control module 260.

The acquisition module 252 can be configured to acquire ultrasound data. In some embodiments, the ultrasound data includes imaging data. In some embodiments, the acquisition module 252 activates the transducers 220 (e.g., less than all of the transducers 220, different subset(s) of the transducers 220, all the transducers 220, etc.) according to whether the ultrasound data meets one or more conditions associated with one or more quality requirements.

The receiving module 254 can be configured to receive ultrasound data.

The transmitting module 256 can be configured to transmit ultrasound data to other device(s) (e.g., a server system, computer device 130, computer device 300, display device 140, and/or other connected devices, etc.).

The analysis module 258 can be configured to analyze whether the data (e.g., imaging data) acquired by the ultrasound device 200 meets one or more conditions associated with quality requirements for an ultrasound scan.

For example, in some embodiments, the one or more conditions include one or more of: a condition that the imaging data includes one or more newly acquired images that meet one or more threshold quality scores; a condition that the imaging data includes one or more newly acquired images that correspond to one or more anatomical planes that match a desired anatomical plane of a target anatomical structure; a condition that the imaging data includes one or more newly acquired images that include one or more landmark/features (or a combination of landmarks/features); a condition that the imaging data includes one or more newly acquired images that include a feature having a particular dimension; a condition that the imaging data supports a prediction that an image meeting one or more requirements would be acquired in the next one or more image frames; a condition that the imaging data supports a prediction that a first change (e.g., an increase by a percentage, or number) in the number of transducer used would support an improvement in the quality score of an image acquired in the next one or more image frames; and/or other analogous conditions.

The transducer control module 260 can be configured to activate (e.g., adjusting) a number of transducers 220 during portions of an ultrasound scan based on a determination that the ultrasound data meets (or does not meet) one or more quality requirements.

For example, in some embodiments, the transducer control module 260 activates a first subset of the transducers 220 during the first portion of an ultrasound scan. In some embodiments, the transducer control module 260 can activate a second subset of the transducers 220, different from the first subset of the transducers, during a second portion of the scan following the first portion of the scan. This can occur when or if the imaging data corresponding to the first portion of the scan meets (or does not meet) one or more quality requirements.

In some embodiments, the transducer control module 260 can control one or more operating modes of the ultrasound device 200. For example, in some embodiments, the ultrasound device 200 is configured to operate in one or more low-power modes. In a respective low-power mode, the transducer control module 260 activates only a subset (e.g., 10%, 15%, 20%, or other preset subsets) of all the available transducers 220 in the ultrasound device 200. In some embodiments, the ultrasound device 200 is configured to operate in a full-power mode. In the full-power mode, the transducer control module 260 activates all the available transducers 220 to acquire a high-quality image.

The device data 280 for the ultrasound device 200 can include, but not be limited to, device settings 282, user settings 284, ultrasound scan data 286, image quality requirements data 288, and/or an atlas 290.

The device settings 282 for the ultrasound device 200 can include, but not be limited to, default options and preferred user settings. In some embodiments, the device settings 282 include imaging control parameters.

For example, in some embodiments, the imaging control parameters include one or more of: a number of transducers that are activated, a power consumption threshold of the probe, an imaging frame rate, a scan speed, a depth of penetration, and other scan parameters that control the power consumption, heat generation rate, and/or processing load of the probe.

The user settings 284 can include, but not be limited to, a preferred gain, depth, zoom, and/or focus settings.

The ultrasound scan data 286 (e.g., imaging data) can be acquired (e.g., detected, measured) by the ultrasound device 200 (e.g., via transducers 220).

In some embodiments, the image quality requirements data 288 include clinical requirements for determining the quality of an ultrasound image.

In some embodiments, the atlas 290 can include anatomical structures of interest. In some embodiments, the atlas 290 includes three-dimensional representations of the anatomical structure of interest (e.g., hip, heart, lung, and/or other anatomical structures).

Each of the above identified executable modules, applications, or sets of procedures may be stored in one or more of the previously mentioned memory devices, and corresponds to a set of instructions for performing a function described above. The above identified modules or programs (i.e., sets of instructions) need not be implemented as separate software programs, procedures, or modules, and thus various subsets of these modules may be combined or otherwise re-arranged in various implementations. In some implementations, the memory 206 stores a subset of the modules and data structures identified above. Furthermore, the memory 206 may store additional modules or data structures not described above. In some embodiments, a subset of the programs, modules, and/or data stored in the memory 206 are stored on and/or executed by a server system, and/or by an external device (e.g., computing device 130 or computing device 300).

FIG. 4 illustrates a block diagram of a computing device 300, in accordance with some embodiments.

In some embodiments, the computing device 300 can comprise a server or control console that is in communication with the ultrasound device 200 (e.g., ultrasound probe). In some embodiments, the computing device 300 can be integrated into the same housing as the ultrasound device 200.

For example, the computing device can comprise a smartphone, tablet device, a gaming console, and/or other portable computing devices. In some embodiments, the computing device 300 may be provided by a combination of components integrated into the same housing as the ultrasound device 200, and a smartphone, tablet device, a gaming console, or other portable computing devices.

The computing device 300 can include one or more processors 302 (e.g., processing units of CPU(s)), one or more network interfaces 304, memory 306, and one or more communication buses 308 for interconnecting these components (sometimes called a chipset), in accordance with some implementations.

In some embodiments, the computing device 300 can include one or more input devices 310 that facilitate user input, such as a keyboard, a mouse, a voice-command input unit or microphone, a touch screen display, a touch-sensitive input pad, a gesture capturing camera, or other input buttons or controls. For example, the computing device 300 can use a microphone and voice recognition or a camera and gesture recognition to supplement and/or replace the keyboard.

Further, in some embodiments, the computing device 300 includes one or more output devices 312 that enable presentation of user interfaces and display content, such as one or more speakers and/or one or more visual displays (e.g., display device 140).

The memory 306 includes high-speed random-access memory, such as DRAM, SRAM, DDR RAM, or other random-access solid-state memory devices; and, optionally, includes non-volatile memory, such as one or more magnetic disk storage devices, one or more optical disk storage devices, one or more flash memory devices, or one or more other non-volatile solid state storage devices. The memory 306, optionally, includes one or more storage devices remotely located from the one or more processors 302. The memory 306, or alternatively the non-volatile memory within the memory 306, includes a non-transitory computer-readable storage medium.

In some implementations, the memory 306 or the non-transitory computer-readable storage medium of the memory 306 (either or both of which may be referred to herein as “memory”) can store various programs, modules, and data structures.

For example, the memory can store programs, modules, and data structures, or a subset or superset thereof, such as an operating system 322, a communication module 323, a user interface module 324, an application 350, and/or a database 380.

The operating system 322 can include procedures for handling various basic system services and for performing hardware dependent tasks. The communication module 323 (e.g., a radio communication module) can be configured to connect to and communicate with other network devices (e.g., a local network, such as a router that provides Internet connectivity, networked storage devices, network routing devices, server systems, computer device 130, ultrasound device 200, and/or other connected devices, etc.) coupled to one or more communication networks via the network interface 304 (e.g., wired or wireless).

In some embodiments, the user interface module 324 can be configured to enable presentation of information (e.g., a graphical user interface for presenting application(s), widgets, websites and web pages thereof, games, audio and/or video content, text, etc.) either at the computing device 300 or another device.

The application 350 can be configured to acquire ultrasound data (e.g., imaging data) from a patient. In some embodiments, the application 350 is used for receiving data (e.g., ultrasound data, imaging data, etc.) acquired via an ultrasound device 200. In some embodiments, the application 350 is used for controlling one or more components of an ultrasound device 200 (e.g., the probe portion, and/or the transducers) and/or other connected devices (e.g., in accordance with a determination that the data meets, or does not meet, certain conditions).

Further, in some embodiments, the application 350 can include an acquisition module 352, a receiving module 354, a transmitting module 354, an analysis module 358, and/or a transducer control module 360.

The acquisition module 352 can be configured to acquire ultrasound data. In some embodiments, the ultrasound data includes imaging data acquired by an ultrasound probe. In some embodiments, the acquisition module 352 activates the transducers 220 (e.g., less than all of the transducers 220, different subset(s) of the transducers 220, all the transducers 220, etc.) according to whether the ultrasound data meets one or more conditions associated with one or more quality requirements. In some embodiments, the acquisition module 352 causes the ultrasound device 200 to activate the transducers 220 (e.g., less than all of the transducers 220, different subset(s) of the transducers 220, all the transducers 220, etc.) according to whether the ultrasound data meets one or more conditions associated with one or more quality requirements.

The receiving module 354 can be configured to receive ultrasound data. In some embodiments, the ultrasound data includes imaging data acquired by an ultrasound probe.

The transmitting module 356 can be configured to transmit ultrasound data (e.g., imaging data) to other device(s) (e.g., a server system, computer device 130, display device 140, ultrasound device 200, and/or other connected devices, etc.).

The analysis module 358 can be configured to analyze whether the data (e.g., imaging data, power consumption data, and other data related to the acquisition process) (e.g., received by the ultrasound probe) meets one or more conditions associated with quality requirements for an ultrasound scan.

For example, in some embodiments, the one or more conditions can include one or more of: a condition that the imaging data includes one or more newly acquired images that meet one or more threshold quality scores; a condition that the imaging data includes one or more newly acquired images that correspond to one or more anatomical planes that match a desired anatomical plane of a target anatomical structure; a condition that the imaging data includes one or more newly acquired images that include one or more landmark/features (or a combination of landmarks/features); a condition that the imaging data includes one or more newly acquired images that include a feature having a particular dimension; a condition that the imaging data supports a prediction that an image meeting one or more requirements would be acquired in the next one or more image frames; a condition that the imaging data supports a prediction that a first change (e.g., an increase by a percentage, or number) in the number of transducer used would support an improvement in the quality score of an image acquired in the next one or more image frames; and/or other analogous conditions.

The transducer control module 360 can be configured to activate (e.g., adjusting, controlling, and/or otherwise modifying one or more operations of the transducers), or causing the ultrasound device 200 to activate (e.g., via the transducer control module 260), a number of transducers 220 during portions of an ultrasound scan based on a determination that the ultrasound data meets (or does not meet) one or more quality requirements.

For example, in some embodiments, the transducer control module 360 activates a first subset of the transducers 220 during the first portion of an ultrasound scan. In some embodiments, the transducer control module 360 activates a second subset of the transducers 220, different from the first subset of the transducers, during a second portion of the scan following the first portion of the scan, when the imaging data corresponding to the first portion of the scan meets (or does not meet) one or more quality requirements.

For example, in some embodiments, the transducer control module 360 controls one or more operating modes of the ultrasound device 200. The ultrasound device 200 is configured to operate in a low-power mode. In the low-power mode, the transducer control module 360 activates only a subset (e.g., 10%, 15%, 20%, etc.) of all the available transducers 220 in the ultrasound device 200. In some embodiments, the ultrasound device 200 is configured to operate in a full-power mode. In the full-power mode, the transducer control module 360 activates all the available transducers 220 to acquire a high-quality image.

The database 380 can include ultrasound scan data 382, image quality requirements data 384, an atlas 386, imaging control parameters 388, ultrasound scan data processing models 390, and/or labeled images 392.

The ultrasound scan data 382 (e.g., imaging data) can be acquired (e.g., detected, measured) by one or more ultrasound probes 200.

The image quality requirements data 384 can include clinical requirements for determining the quality of an ultrasound image.

In some embodiments, the atlas 386 can include anatomical structures of interest. In some embodiments, the atlas 386 includes three-dimensional representations of the anatomical structure of interest (e.g., hip, heart, or lung).

In some embodiments, the imaging control parameters 388 can include one or more of: a number of transducers that are activated; a power consumption threshold of the probe; an imaging frame rate; a scan speed; a depth of penetration; and/or other scan parameters that control the power consumption, heat generation rate, and/or processing load of the probe.

The ultrasound scan data processing models 390 can be configured to process ultrasound data. For example, in some embodiments, the ultrasound scan data processing models 390 are trained neural network models that are trained to determine whether an ultrasound image meets quality requirements corresponding to a scan type, or trained to output an anatomic plane corresponding to an anatomical structure of an ultrasound image, or trained to predict, based on a sequence of ultrasound images and their quality scores, whether a subsequent frame to be acquired by an ultrasound probe will contain certain anatomical structures and/or landmarks of interest.

The labeled images 392 (e.g., a databank of images), can include images for training the models that are used for processing new ultrasound data, and/or new images that have been or need to be processed. In some embodiments, the labeled images 392 are images of anatomical structures that have been labeled with their respective identifiers and relative positions.

Each of the above identified elements may be stored in one or more of the memory devices described herein, and corresponds to a set of instructions for performing the functions described above. The above identified modules or programs need not be implemented as separate software programs, procedures, modules or data structures, and thus various subsets of these modules may be combined or otherwise re-arranged in various embodiments. In some embodiments, the memory 306, optionally, stores a subset of the modules and data structures identified above. Furthermore, the memory 306 optionally stores additional modules and data structures not described above. In some embodiments, a subset of the programs, modules, and/or data stored in the memory 306 are stored on and/or executed by the ultrasound probe 200.

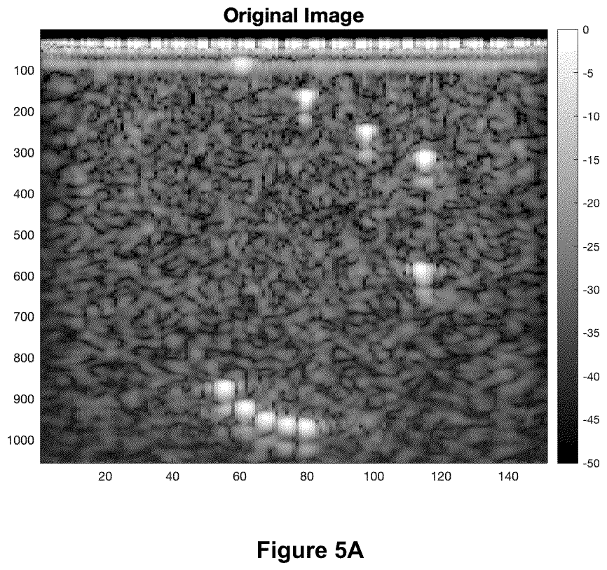

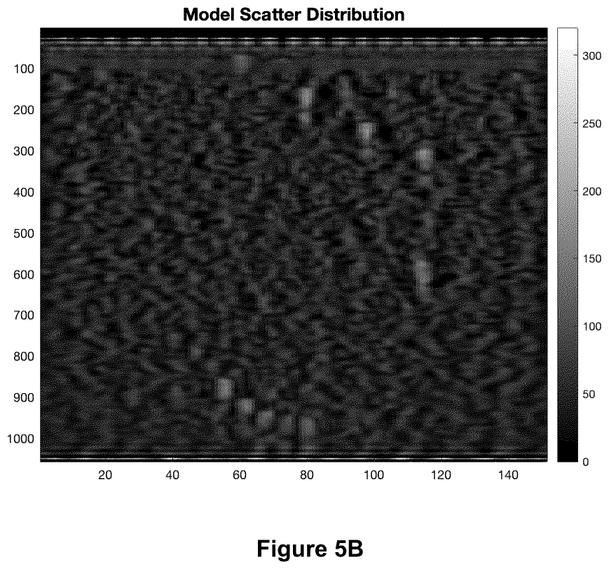

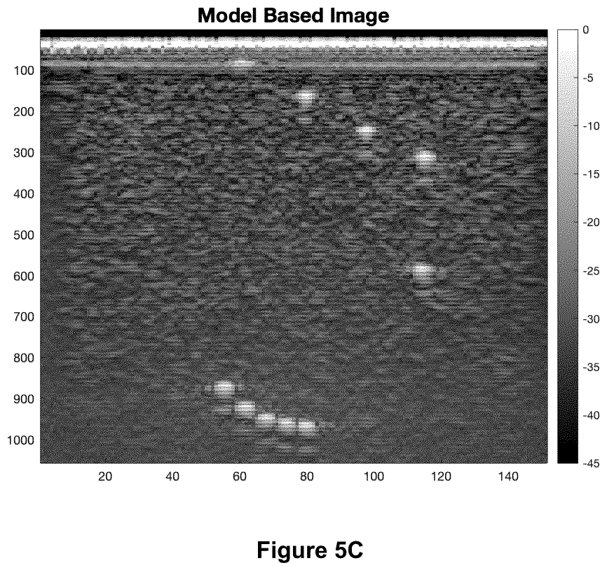

FIGS. 5A-5C illustrate exemplary actual ultrasound image, simulated ultrasound image, and a final, synthesized, ultrasound image, that may be created in accordance with an embodiment. FIG. 5B is a simulated ultrasound image. As an example, a simulation may be created using a model scatterer distribution as shown in FIG. 5B. However, as disclosed, other models and methods may be used. As shown, FIG. 5C is an improved version of FIG. 5A, which is improved by the simulated ultrasound image in FIG. 5B.

The foregoing description, for purpose of explanation, has been described with reference to specific implementations. However, the illustrative discussions above are not intended to be exhaustive or to limit the scope of the claims to the precise forms disclosed. Many modifications and variations are possible in view of the above teachings. The implementations are chosen in order to best explain the principles underlying the claims and their practical applications, to thereby enable others skilled in the art to best use the implementations with various modifications as are suited to the particular uses contemplated.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A method of forming a final ultrasound image, comprising: at a computing device of an ultrasound imaging device: receiving an actual ultrasound image from the ultrasound imaging device, the actual ultrasound image being formed using parameters; creating a synthetic ultrasound image using a simulation model based on the parameters; and forming the final ultrasound image based on the synthetic ultrasound image and the actual ultrasound image.

Clause 2. The method of Clause 1, wherein the receiving the actual ultrasound image from the ultrasound imaging device comprises receiving the actual ultrasound image from the ultrasound imaging device at a first imaging frequency.

Clause 3. The method of any of the preceding Clauses, wherein the parameters include a scatter intensity and distribution pattern used to form the actual ultrasound image.

Clause 4. The method of Clause 3, wherein the scatter intensity and distribution pattern is a distribution of an amount of ultrasound energy that is scattered or reflected back from an anatomy of interest.

Clause 5. The method of Clause 3, wherein the synthesizing further comprises training the simulation model by comparing the actual ultrasound image and an initial simulated ultrasound image.

Clause 6. The method of Clause 3, wherein the synthesizing further comprises training the simulation model by comparing the scatterer intensity and distribution pattern to expected templates of scatterer intensity and distribution pattern for an anatomy of interest to reduce artifacts.

Clause 7. The method of Clause 6, wherein the artifacts include noise.

Clause 8. The method of any of the preceding Clauses, wherein the simulation model is a forward propagation model.

Clause 9. The method of any of the preceding Clauses, wherein the simulation model is an artificial intelligence (AI) model.

Clause 10. The method of any of the preceding Clauses, wherein the simulation model is a real-time ultrasound model based on a real-time simulation of ultrasound data.

Clause 11. The method of any of the preceding Clauses, wherein the simulation model is a forward propagation real-time ultrasound model.

Clause 12. The method of any of the preceding Clauses, wherein the simulation model is a spatial-temporal model.

Clause 13. The method of Clause 12, wherein the spatial-temporal model is used to create lateral flow that moves in a direction perpendicular relative to an ultrasound beam.

Clause 14. The method of Clause 12, wherein the spatial-temporal model is used to create anatomic flow that moves in a direction that is neither strictly parallel relative to nor strictly perpendicular relative to an ultrasound beam.

Clause 15. The method of any of the preceding Clauses, wherein the simulation model is based on an underlying anatomy of the actual ultrasound image.

Clause 16. The method of any of the preceding Clauses, wherein the simulation model is specific to an underlying anatomy of the actual ultrasound image.

Clause 17. The method of any of the preceding Clauses, wherein the parameters include transducer parameters and ultrasound imaging parameters to form the actual ultrasound image.

Clause 18. The method of Clause 17, wherein the synthesizing further comprises adapting the parameters used to form the actual ultrasound image to minimize differences between the actual ultrasound image and an initial simulated ultrasound image.

Clause 19. The method of Clause 18, wherein the parameters include a scatter intensity and wherein the synthesizing further comprises adapting the scatterer intensity and distribution pattern to minimize differences between the actual ultrasound image and the initial simulated ultrasound image.

Clause 20. The method of Clause 19, wherein the synthesizing further comprises using the adapted scatterer intensity and distribution pattern to form the simulated ultrasound image.

Clause 21. The method of Clause 17, wherein the synthesizing further comprises adapting the transducer parameters to reduce reverb to minimize differences between the actual ultrasound image and an initial simulated ultrasound image.

Clause 22. The method of Clause 17, wherein the synthesizing further comprises adapting a lens thickness to reduce reverb to minimize differences between the actual ultrasound image and an initial simulated ultrasound image.

Clause 23. The method of any of the preceding Clauses, wherein the final ultrasound image is at a desired resolution different from a resolution of the actual ultrasound image.

Clause 24. The method of any of the preceding Clauses, wherein the final ultrasound image is at one or more frequencies different from a frequency of the actual ultrasound image.

Clause 25. The method of any of the preceding Clauses, wherein the final ultrasound image is at one or more apertures different from an aperture of the actual ultrasound image.

Clause 26. The method of any of the preceding Clauses, wherein the final ultrasound image is at one or more directions different from a direction of the actual ultrasound image.

Clause 27. The method of any of the preceding Clauses, wherein the synthesizing comprises improving penetration of the actual ultrasound image to form the final ultrasound image.

Clause 28. The method of any of the preceding Clauses, wherein the synthesizing comprises improving resolution of the actual ultrasound image to form the final ultrasound image.

Clause 29. The method of any of the preceding Clauses, wherein the synthesizing comprises improving speckle of the actual ultrasound image to form the final ultrasound image.

Clause 30. The method of any of the preceding Clauses, wherein the synthesizing comprises using a higher frame rate than the actual ultrasound image to form the final ultrasound image.

Clause 31. The method of Clause 2, wherein the synthesizing comprises displaying the final ultrasound image at a second imaging frequency.

Clause 32. The method of Clause 31, wherein the second imaging frequency is greater than the first imaging frequency, the actual ultrasound image is at a first spatial resolution, the final ultrasound image is at a second spatial resolution, and the second spatial resolution is higher than the first spatial resolution.

Clause 33. The method of any of the preceding Clauses, wherein the synthesizing comprises reducing or eliminating reverb noise of the final ultrasound image based on the simulation model of an imaging anatomy.

Clause 34. The method of any of the preceding Clauses, wherein the synthesizing comprises removing unwanted aspects from the simulated ultrasound image.

Clause 35. The method of any of the preceding Clauses, wherein the synthesizing comprises removing artifacts that arise from a transducer from the simulated ultrasound image.

Clause 36. The method of any of the preceding Clauses, wherein the synthesizing comprises improving a penetration depth of the simulated ultrasound image at high imaging frequencies.

Clause 37. The method of any of the preceding Clauses, wherein the synthesizing comprises training the simulation model with time to create a spatial-temporal model.

Clause 38. The method of any of the preceding Clauses, wherein the synthesizing comprises improving a doppler image quality of the final ultrasound image.

Clause 39. The method of any of the preceding Clauses, wherein the synthetic ultrasound is a scatterer model.

Clause 40. The method of any of the preceding Clauses, wherein the synthesizing comprises improving a needle detection of the final ultrasound image.

Clause 41. The method of Clause 1 wherein the synthesizing comprises rendering the final ultrasound image using motion mode.

Clause 42. The method of any of the preceding Clauses, wherein the synthesizing comprises training the simulation model by automatically identifying and adapting imaging presets by comparing one or more of the simulation models with one or more templates of stored models.

Clause 43. A method of processing an ultrasound image, comprising: receiving the ultrasound image from an anatomy of interest; training a model based on the anatomy of interest by minimizing a difference between sample ultrasound images and expected ultrasound images; and generating an enhanced ultrasound image via the trained model from the ultrasound image.

Clause 44. An ultrasound imaging device, comprising: a computing circuit; one or more processors; memory; and one or more programs stored in the memory and configured for execution by the one or more processors, the one or more programs comprising instructions for: receiving an actual ultrasound image from the ultrasound imaging device, the actual ultrasound image being formed using parameters; creating a synthetic ultrasound image using a simulation model based on the parameters; and forming a final ultrasound image based on the synthetic ultrasound image and the actual ultrasound image.

Clause 45. A non-transitory computer readable storage medium storing one or more programs that, when executed by a computing device having one or more processors and memory, cause the computing device to perform operations comprising: receiving an actual ultrasound image from an ultrasound imaging device, the actual ultrasound image being formed using parameters; creating a synthetic ultrasound image using a simulation model based on the parameters; and forming a final ultrasound image based on the synthetic ultrasound image and the actual ultrasound image.

Clause 46. The non-transitory computer readable storage comprising any steps disclosed in any of the preceding Clauses.

Clause 47. A method comprising any steps disclosed in any of the preceding Clauses.

Clause 48. A system comprising one or more devices configured to perform any of the methods disclosed in any of the preceding Clauses.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

It will also be understood that, although the terms first, second, etc., are, in some instances, used herein to describe various elements, these elements should not be limited by these terms. These terms are only used to distinguish one element from another. For example, a first electronic device could be termed a second electronic device, and, similarly, a second electronic device could be termed a first electronic device, without departing from the scope of the various described implementations. The first electronic device and the second electronic device are both electronic devices, but they are not necessarily the same electronic device.

The terminology used in the description of the various described implementations herein is for the purpose of describing particular implementations only and is not intended to be limiting. As used in the description of the various described implementations and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

As used herein, the term “if” is, optionally, construed to mean “when” or “upon” or “in response to determining” or “in response to detecting” or “in accordance with a determination that,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event]” or “in accordance with a determination that [a stated condition or event] is detected,” depending on the context.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

As used herein, the term “about” is relative to the actual value stated, as will be appreciated by those of skill in the art, and allows for approximations, inaccuracies and limits of measurement under the relevant circumstances. In one or more aspects, the terms “about,” “substantially,” and “approximately” may provide an industry-accepted tolerance for their corresponding terms and/or relativity between items, such as a tolerance of from less than one percent to 10% percent of the actual value stated, and other suitable tolerances.

As used herein, the term “comprising” indicates the presence of the specified integer(s), but allows for the possibility of other integers, unspecified. This term does not imply any particular proportion of the specified integers. Variations of the word “comprising,” such as “comprise” and “comprises,” have correspondingly similar meanings.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

Although the detailed description contains many specifics, these should not be construed as limiting the scope of the subject technology but merely as illustrating different examples and aspects of the subject technology. It should be appreciated that the scope of the subject technology includes other embodiments not discussed in detail above. Various other modifications, changes and variations may be made in the arrangement, operation and details of the method and apparatus of the subject technology disclosed herein without departing from the scope of the present disclosure. In addition, it is not necessary for a device or method to address every problem that is solvable (or possess every advantage that is achievable) by different embodiments of the disclosure in order to be encompassed within the scope of the disclosure. The use herein of “can” and derivatives thereof shall be understood in the sense of “possibly” or “optionally” as opposed to an affirmative capability.

What is claimed is:

1. A method of forming a final ultrasound image, comprising, at a computing device of an ultrasound imaging device:
receiving an actual ultrasound image from the ultrasound imaging device, the actual ultrasound image being formed using parameters;
creating a synthetic ultrasound image using a simulation model based on the parameters; and
forming the final ultrasound image based on the synthetic ultrasound image and the actual ultrasound image.

2. The method of claim 1, wherein the receiving the actual ultrasound image from the ultrasound imaging device comprises receiving the actual ultrasound image from the ultrasound imaging device at a first imaging frequency.

3. The method of claim 1, wherein the parameters include at least one of a scatterer intensity or a distribution pattern.

4. The method of claim 3, wherein the synthesizing further comprises training the simulation model by comparing the actual ultrasound image and an initial simulated ultrasound image.

5. The method of claim 1, wherein the simulation model is an artificial intelligence (AI) model.

6. The method of claim 1, wherein the simulation model is a real-time ultrasound model based on a real-time simulation of ultrasound data.

7. The method of claim 1, wherein the parameters include transducer parameters and ultrasound imaging parameters to form the actual ultrasound image.

8. The method of claim 7, wherein the synthesizing further comprises adapting the parameters used to form the actual ultrasound image to minimize differences between the actual ultrasound image and an initial simulated ultrasound image.

9. The method of claim 8, wherein the synthesizing further comprises adapting the scatterer intensity and distribution pattern to minimize differences between the actual ultrasound image and the initial simulated ultrasound image.

10. The method of claim 9, wherein the synthesizing further comprises using the adapted scatterer intensity and distribution pattern to form the simulated ultrasound image.

11. The method of claim 2, wherein the synthesizing comprises displaying the final ultrasound image at a second imaging frequency.

12. The method of claim 11, wherein the second imaging frequency is greater than the first imaging frequency, the actual ultrasound image is at a first spatial resolution, the final ultrasound image is at a second spatial resolution, and the second spatial resolution is higher than the first spatial resolution.

13. The method of claim 1, wherein the synthesizing comprises reducing or eliminating reverb noise of the final ultrasound image based on the simulation model of an imaging anatomy.

14. The method of claim 1, wherein the synthesizing comprises improving a penetration depth of the simulated ultrasound image at high imaging frequencies.

15. The method of claim 1, wherein the synthesizing comprises training the simulation model with time to create a spatial-temporal model.

16. The method of claim 1, wherein the synthesizing comprises improving a doppler image quality of the final ultrasound image.

17. The method of claim 1, wherein the synthesizing comprises improving a needle detection of the final ultrasound image.

18. The method of claim 1, wherein the synthesizing comprises rendering the final ultrasound image using motion mode.

19. The method of claim 1, wherein the synthesizing comprises training the simulation model by automatically identifying and adapting imaging presets by comparing one or more of the simulation models with one or more templates of stored models.

20. An ultrasound imaging device, comprising:
a computing circuit;
one or more processors;
memory; and
one or more programs stored in the memory and configured for execution by the one or more processors, the one or more programs comprising instructions for:
receiving an actual ultrasound image from the ultrasound imaging device, the actual ultrasound image being formed using parameters; and
creating a synthetic ultrasound image using a simulation model based on the parameters; and
forming the final ultrasound image based on the synthetic ultrasound image and the actual ultrasound image.
